(19) United States
(12) Patent Application Publication
(10) Pub. No.: US 2019/0090778 A1
(43) Pub. Date: Mar. 28, 2019

(54) CAPSULE ENDOSCOPE POSITION DETECTION METHOD AND CAPSULE ENDOSCOPE POSITION DETECTION APPARATUS

(71) Applicant: OLYMPUS CORPORATION, Tokyo (JP)

(72) Inventors: Yoshinori IKEDA, Sagamihara-shi (JP); Shinichi NAKAJIMA, Tokyo (JP); Akihiro KUBOTA, Tokyo (JP)

(73) Assignee: OLYMPUS CORPORATION, Tokyo (JP)

(21) Appl. No.: 16/198,050

(22) Filed: Nov. 21, 2018

Related U.S. Application Data
(63) Continuation of application No. PCT/JP2016/066491, filed on Jun. 2, 2016.

(51) Int. Cl. A61B 5/06; A61B 1/04; A61B 1/00

(52) U.S. Cl. CPC A61B 5/061; A61B 1/00016; A61B 1/041

(57) ABSTRACT

A capsule endoscope position detection method includes first to third steps. The method uses at least four antennas configured to receive signals wirelessly transmitted from a capsule endoscope inside a subject. In the first step, a calculation device calculates a distance difference between a first distance and a second distance on the basis of the signals received by two of the at least four antennas. The first distance is a distance between one of the two antennas and a capsule endoscope. The second distance is a distance between the other of the two antennas and the capsule endoscope. In the second step, the calculation device selects at least three distance differences among the distance differences calculated in the first step. In the third step, the calculation device calculates a Z coordinate of the capsule endoscope on the basis of the selected distance difference.

### BACKGROUND OF THE INVENTION

### Field of the Invention

The present invention relates to a capsule endoscope position detection method and a capsule endoscope position detection apparatus.

This application is a continuation application based on International Patent Application No. PCT/JP 2016/066491 filed on Jun. 2, 2016, the content of which is incorporated herein by reference.

### Description of Related Art

A capsule endoscope for observing the inside of a living body is used. Since the capsule endoscope may be moved by peristaltic motion inside the body, it is necessary to recognize an image capturing position inside the body. For this reason, it is important to measure the position of a capsule endoscope.

A method of measuring the position of a capsule endoscope is disclosed in Japanese Unexamined Patent Application, First Publication No. 2009-000401. In the method disclosed in Japanese Unexamined Patent Application, First Publication No. 2009-000401, signals transmitted at the same time from the capsule endoscope are received by a plurality of antennas. The internal clocks of chips on which the antennas are disposed are synchronized with each other. A phase difference between a signal receiving time of each antenna and the internal clock is detected. A difference in the phase differences corresponding to each of the two antennas corresponds to a difference in signal receiving times of the two antennas. For example, a phase difference corresponding to an antenna A may be Pda and a phase difference corresponding to an antenna B may be Pdb. The phase difference Pda is a phase difference between a signal receiving time using the antenna A and an internal clock. The phase difference Pdb is a phase difference between a signal receiving time using the antenna B and an internal clock.

A difference between the phase difference Pda and the phase difference Pdb corresponds to a difference between a time when a radio wave from the capsule endoscope is received by the antenna A and a time when a radio wave from the capsule endoscope is received by the antenna B. That is, a difference between the phase difference Pda and the phase difference Pdb corresponds to a distance difference between a first distance and a second distance. The first distance is a distance between the antenna A and the capsule endoscope. The second distance is a distance between the antenna B and the capsule endoscope. The distance differences for a plurality of combinations of two antennas are calculated. The position of the capsule endoscope is calculated on the basis of the distance difference corresponding to each combination.

### SUMMARY OF THE INVENTION

According to a first aspect of the present invention, a capsule endoscope position detection method includes a first step, a second step, and a third step. The method uses at least four antennas configured to receive signals wirelessly transmitted from a capsule endoscope inside a subject. In the first step, the calculation device calculates a distance difference between a first distance and a second distance on the basis of the signals wirelessly transmitted from the capsule endoscope and received by two of the at least four antennas. The at least four antennas are disposed on a first plane. The first plane is substantially parallel to a second plane. The second plane includes X and Y axes in an orthogonal coordinate system including X, Y, and Z axes. The first distance is a distance between one of the two antennas and the capsule endoscope. The second distance is a distance between the other of the two antennas and the capsule endoscope. In the first step, the distance difference is calculated for each of at least four combinations of the two antennas. In the second step, the calculation device selects at least three distance differences on the basis of the magnitude of the distance differences calculated in the first step. In the third step, the calculation device calculates a Z coordinate of the capsule endoscope on the basis of the distance differences selected in the second step.

According to the second aspect of the present invention, in the second step of the first aspect, the calculation device may select three large distance differences from the top in the order of magnitude of the distance differences for the at least four combinations.

According to a third aspect of the present invention, in the second step of the first aspect, the calculation device may select at least three distance differences which are equal to or larger than a predetermined value among the distance differences for the at least four combinations.

According to a fourth aspect of the present invention, in the third aspect, the predetermined value may be an average value of the distance differences for the at least four combinations or a median value of the distance differences for the at least four combinations.

According to a fifth aspect of the present invention, in the first aspect, a first average value may be larger than a second average value. The first average value is an average value of the at least three distance differences selected in the second step. The second average value is an average value of the distance differences for the at least four combinations.

According to a sixth aspect of the present invention, in any one of the first to fifth aspects, the capsule endoscope position detection method may further include a fourth step, a fifth step, and a sixth step. In the fourth step, the calculation device calculates the distance difference on the basis of the signals transmitted from the capsule endoscope and received by the two antennas of which X coordinates are different among the at least four antennas. In the fourth step, the distance difference for each of at least four combinations of the two antennas is calculated. In the fifth step, the calculation device selects at least three distance differences among the distance differences calculated in the fourth step. In the sixth step, the calculation device calculates an X coordinate of the capsule endoscope on the basis of the distance differences selected in the fifth step.

According to a seventh aspect of the present invention, in any one of the first to fifth aspects, the capsule endoscope position detection method may further include a fourth step, a fifth step, and a sixth step. In the fourth step, the calculation device calculates the distance difference on the basis of the signals transmitted from the capsule endoscope and received by the two antennas of which Y coordinates are different among the at least four antennas. In the fourth step, the distance difference for each of at least four combinations of the two antennas is calculated. In the fifth step, the calculation device selects at least three distance differences among the distance differences calculated in the fourth step. In the sixth step, the calculation device calculates a Y coordinate of the capsule endoscope on the basis of the distance differences selected in the fifth step.

According to eighth and ninth aspects of the present invention, in the sixth or seventh aspect, in the fifth step, the calculation device may select the three small distance differences from the bottom in the order of magnitude of the distance differences for the at least four combinations.

According to tenth and eleventh aspects of the present invention, in the fifth step of the sixth or seventh aspect, the calculation device may select at least three distance differences in which the distance difference is equal to or smaller than a predetermined value among the distance differences for the at least four combinations.

According to twelfth and thirteenth aspects of the present invention, in the tenth or eleventh aspect, the predetermined value may be an average value of the distance differences for the at least four combinations or a median value of the distance differences for the at least four combinations.

According to fourteenth and fifteenth aspects of the present invention, in the sixth or seventh aspect, a third average value may be smaller than a fourth average value. The third average value is an average value of the at least three distance differences selected in the fifth step. The fourth average value is an average value of the distance differences for the at least four combinations.

According to a sixteenth aspect of the present invention, in the first step of any one of the first to eleventh aspects, the calculation device may calculate the distance difference on the basis of a time difference or a phase difference. The time difference is a difference in time when the same signals transmitted from the capsule endoscope are received by the two antennas. The phase difference is a phase difference in the signals received by the two antennas when the same signals transmitted from the capsule endoscope are received by the two antennas.

According to a seventeenth aspect of the present invention, in the fourth step of any one of the sixth to eleventh aspects, the calculation device may calculate the distance difference on the basis of a time difference or a phase difference. The time difference is a difference in time when the same signals transmitted from the capsule endoscopes are received by the two antennas. The phase difference is a phase difference in the signals received by the two antennas when the same signals transmitted from the capsule endoscope are received by the two antennas.

According to an eighteenth aspect of the present invention, in the first step of any one of the first to eleventh aspects, the calculation device may calculate the distance difference on the basis of a signal strength of the signals received by the two antennas when the same signals transmitted from the capsule endoscope are received by the two antennas.

According to a nineteenth aspect of the present invention, in the fourth step of any one of the sixth to eleventh aspects, the calculation device may calculate the distance difference on the basis of a signal strength of the signals received by the two antennas when the same signals transmitted from the capsule endoscope are received by the two antennas.

According to a twentieth aspect of the present invention, a capsule endoscope position detection apparatus includes at least four antennas, an antenna selection circuit, and a calculation device. The at least four antennas receive signals wirelessly transmitted from a capsule endoscope inside a subject. The antenna selection circuit sequentially selects one or two antennas from the at least four antennas. The at least four antennas are disposed on the first plane. A first plane is substantially parallel to the second plane. A second plane includes X and Y axes in an orthogonal coordinate system including X, Y, and Z axes. In a first step, the calculation device calculates a distance difference between a first distance and a second distance on the basis of signals received by two antennas simultaneously or sequentially selected by the antenna selection circuit. The first distance is a distance between one of the two antennas and the capsule endoscope. The second distance is a distance between the other of the two antennas and the capsule endoscope. In the first step, the distance difference is calculated for each of at least four combinations of the two antennas. In a second step, the calculation device selects at least three distance differences in the order of magnitude of the distance differences calculated in the first step. In a third step, the calculation device calculates a Z coordinate of the capsule endoscope on the basis of the distance differences selected in the second step.

### BRIEF DESCRIPTION OF THE DRAWINGS

FIG. 1 is a block diagram showing a hardware configuration of a capsule endoscope position detection apparatus according to an embodiment of the present invention.

FIG. 2 is a reference diagram showing an orthogonal coordinate system according to the embodiment of the present invention.

FIG. 3 is a block diagram showing a hardware configuration of a capsule endoscope position detection apparatus according to a first modified example of the embodiment of the present invention.

FIG. 4 is a block diagram showing a hardware configuration of a capsule endoscope position detection apparatus according to a second modified example of the embodiment of the present invention.

FIG. 5 is a flowchart showing a sequence of detecting a position of a capsule endoscope according to the embodiment of the present invention.

FIG. 6 is a reference diagram showing estimation accuracy of a Z coordinate in the embodiment of the present invention.

FIG. 7 is a reference diagram showing estimation accuracy of a Z coordinate in the embodiment of the present invention.

FIG. 8 is a reference diagram showing estimation accuracy of a Z coordinate in the embodiment of the present invention.

FIG. 9 is a reference diagram showing estimation accuracy of a Z coordinate in the embodiment of the present invention.

FIG. 10 is a reference diagram showing estimation accuracy of an X coordinate in the embodiment of the present invention.

FIG. 11 is a reference diagram showing estimation accuracy of an X coordinate in the embodiment of the present invention.

FIG. 12 is a reference diagram showing estimation accuracy of an X coordinate in the embodiment of the present invention.

FIG. 13 is a reference diagram showing estimation accuracy of an X coordinate in the embodiment of the present invention.

FIG. 14 is a reference diagram showing a distance difference of each of combinations of two antennas according to the embodiment of the present invention.

### DETAILED DESCRIPTION OF THE INVENTION

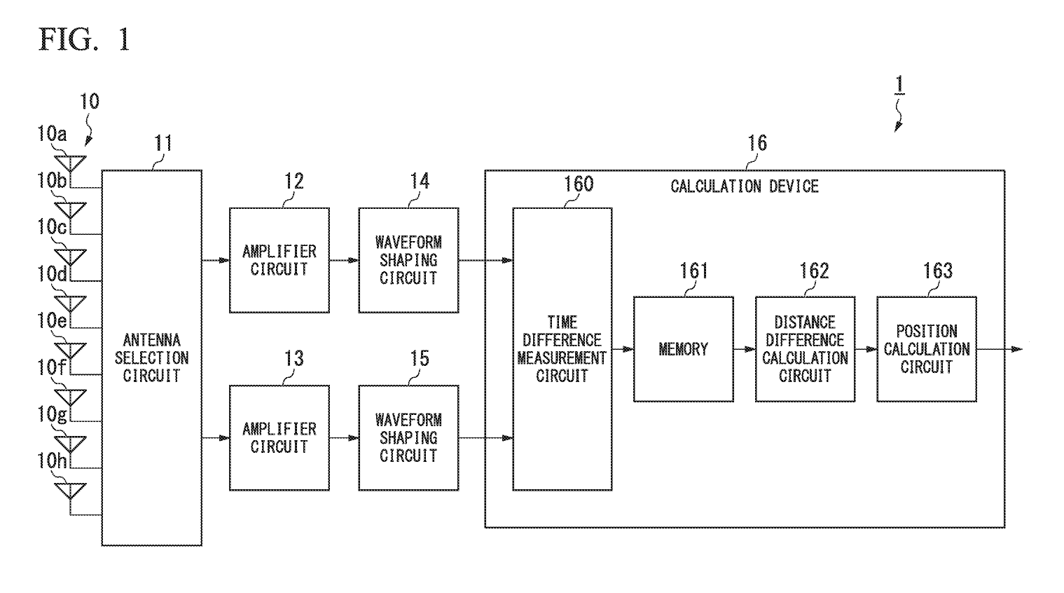

Referring to the drawings, an embodiment of the present invention will be described. FIG. 1 shows a hardware configuration of a capsule endoscope position detection apparatus 1 according to the embodiment of the present invention.

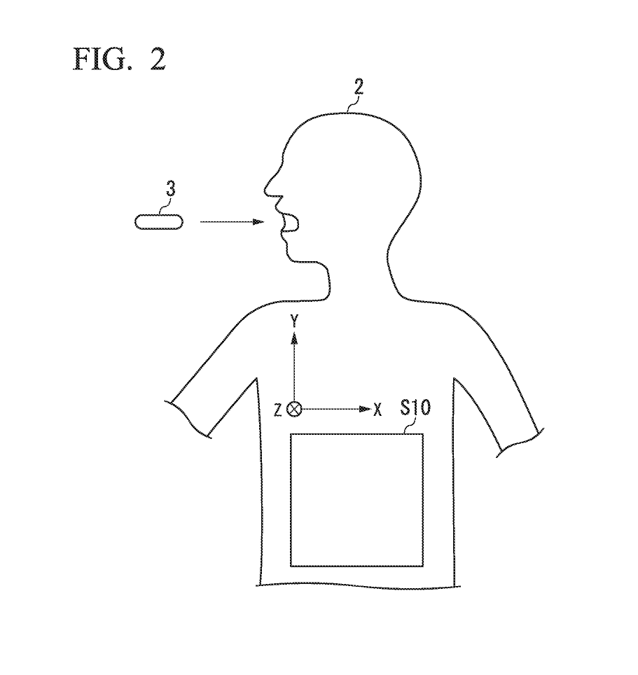

A schematic configuration of the capsule endoscope position detection apparatus 1 will be described. The capsule endoscope position detection apparatus 1 includes at least four antennas 10, an antenna selection circuit 11, and a calculation device 16. At least four antennas 10 receive a signal wirelessly transmitted from a capsule endoscope 3 (FIG. 2) inside a subject 2 (FIG. 2). The antenna selection circuit 11 sequentially selects two antennas 10 from at least four antennas 10. At least four antennas 10 are disposed on a first plane. The first plane is substantially parallel to a second plane. The second plane includes X and Y axes in an orthogonal coordinate system including X, Y, and Z axes. In a first step, the calculation device 16 calculates a distance difference between a first distance and a second distance on the basis of signals received by two antennas 10 selected by the antenna selection circuit 11. The first distance is a distance between one of two antennas 10 and the capsule endoscope 3. The second distance is a distance between the other of two antennas 10 and the capsule endoscope 3. A distance difference for each of at least four combinations of two antennas is calculated. In a second step, the calculation device 16 selects at least three distance differences from the distance differences calculated in the first step. In a third step, the calculation device 16 calculates a Z coordinate of the capsule endoscope on the basis of the distance differences selected in the second step.

A detailed configuration of the capsule endoscope position detection apparatus 1 will be described. As shown in FIG. 1, the capsule endoscope position detection apparatus 1 includes eight antennas 10, the antenna selection circuit 11, an amplifier circuit 12, an amplifier circuit 13, a waveform shaping circuit 14, a waveform shaping circuit 15, and a calculation device 16.

FIG. 2 shows an orthogonal coordinate system in the embodiment of the present invention. The X axis is parallel to the left and right direction of the subject 2 which is a living body. The Y axis is parallel to the up and down direction of the subject 2. The X axis may be parallel to the up and down direction of the subject 2 and the Y axis may be parallel to the left and right direction of the subject 2. The X axis and the Y axis exist within a plane substantially parallel to a front surface of the subject 2. The Z axis is parallel to the front and rear direction of the subject 2. The capsule endoscope 3 is disposed inside the subject 2. Eight antennas 10 are disposed outside the subject 2. Eight antennas 10 are disposed on a plane S10 (a first plane). The plane S10 is substantially parallel to an XY plane (a second plane) including the X axis and the Y axis.

Eight antennas 10 include an antenna 10a, an antenna 10b, an antenna 10c, an antenna 10d, an antenna 10e, an antenna 10f, an antenna 10g, and an antenna 10h. In description in which it is not necessary to distinguish the antennas, the antenna 10 is used. In description in which it is necessary to distinguish the antennas, each of the antennas 10a to 10h is used. The number of the antennas 10 need not be eight. For example, a signal transmitted from the capsule endoscope 3 may be a signal which includes an image captured by the capsule endoscope 3 or a signal for measuring a position. After a signal is transmitted from the capsule endoscope 3, the antenna 10 receives the signal from the capsule endoscope 3.

The antenna selection circuit 11 sequentially selects two antennas 10 from eight antennas 10. The antenna selection circuit 11 outputs a signal output from one of two antennas selected at the same time to the amplifier circuit 12 and outputs a signal which is output from the other of two antennas selected at the same time to the amplifier circuit 13. The amplifier circuit 12 and the amplifier circuit 13 (the amplifiers) amplify the signal output from the antenna selection circuit 11. The amplifier circuit 12 outputs the amplified signal to the waveform shaping circuit 14 and the amplifier circuit 13 outputs the amplified signal to the waveform shaping circuit 15. The waveform shaping circuit 14 and the waveform shaping circuit 15 (the filters) remove a signal having a frequency other than a predetermined frequency band from each of the signals output from the amplifier circuit 12 and the amplifier circuit 13. The amplifier circuit 12 and the amplifier circuit 13 output a processed signal to the calculation device 16.

The calculation device 16 includes a time difference measurement circuit 160, a memory 161, a distance difference calculation circuit 162, and a position calculation circuit 163. The time difference measurement circuit 160 measures a time difference. The time difference is a difference in time when the same signals transmitted from the capsule endoscope 3 are received by two antennas 10 selected by the antenna selection circuit 11. That is, the time difference is a difference between the time at which a signal is received by one of two antennas 10 and the time at which a signal is received by the other of two antennas 10. The time difference is a difference in time when a radio wave transmitted from the capsule endoscope 3 reaches two antennas 10. The time difference measurement circuit 160 measures a time difference for each of combinations of two antennas 10 selected by the antenna selection circuit 11. The memory 161 stores the time difference measured by the time difference measurement circuit 160.

The distance difference calculation circuit 162 calculates the distance difference between the first distance and the second distance on the basis of the time difference stored in the memory 161. The first distance is a distance between one of two antennas 10 selected by the antenna selection circuit 11 and the capsule endoscope 3. The second distance is a distance between the other of two antennas 10 selected by the antenna selection circuit 11 and the capsule endoscope 3. The number of combinations of two antennas 10 in eight antennas 10 is twenty-eight. For example, the distance difference calculation circuit 162 may calculate the distance difference for each of twenty-eight combinations. The distance difference calculation circuit 162 has only to calculate the distance difference for each of at least four combinations.

The position calculation circuit 163 calculates the position of the capsule endoscope 3 on the basis of the distance differences calculated by the distance difference calculation circuit 162. The position calculation circuit 163 calculates an X coordinate, a Y coordinate, and a Z coordinate of the capsule endoscope 3. The position calculation circuit 163 selects at least three distance differences from the distance differences calculated by the distance difference calculation circuit 162 at the time of calculating the coordinates. The position calculation circuit 163 calculates the coordinates on the basis of at least three selected distance differences.

For example, the calculation device 16 includes one or more processors. For example, the processor is constituted as a CPU (Central Processing Unit). One or a plurality of the time difference measurement circuit 160, the distance difference calculation circuit 162, and the position calculation circuit 163 are constituted as one or more processors. One or a plurality of the time difference measurement circuit 160, the distance difference calculation circuit 162, and the position calculation circuit 163 may be constituted as one or more application specific integrated circuits (ASIC) or field programmable gate arrays (FPGA).

For example, the functions of the time difference measurement circuit 160, the distance difference calculation circuit 162, and the position calculation circuit 163 can be realized as a function of software by the processor reading a program including a command for defining the operation of the processor. This program may be provided by, for example, a “computer readable storage medium” such as a flash memory. Further, the above-described program may be transmitted from a computer including a storage device storing this program to the calculation device 16 via a transmission medium or a transmission wave in the transmission medium. The “transmission medium” that transmits the program is a medium that has a function of transmitting information like a network (a communication network) such as the Internet or a communication line such as a telephone line. Further, the above-described program may realize a part of the above-described function. Furthermore, the above-described program may be a difference file (a difference program) which can realize the above-described function in combination with a program stored in the computer in advance.

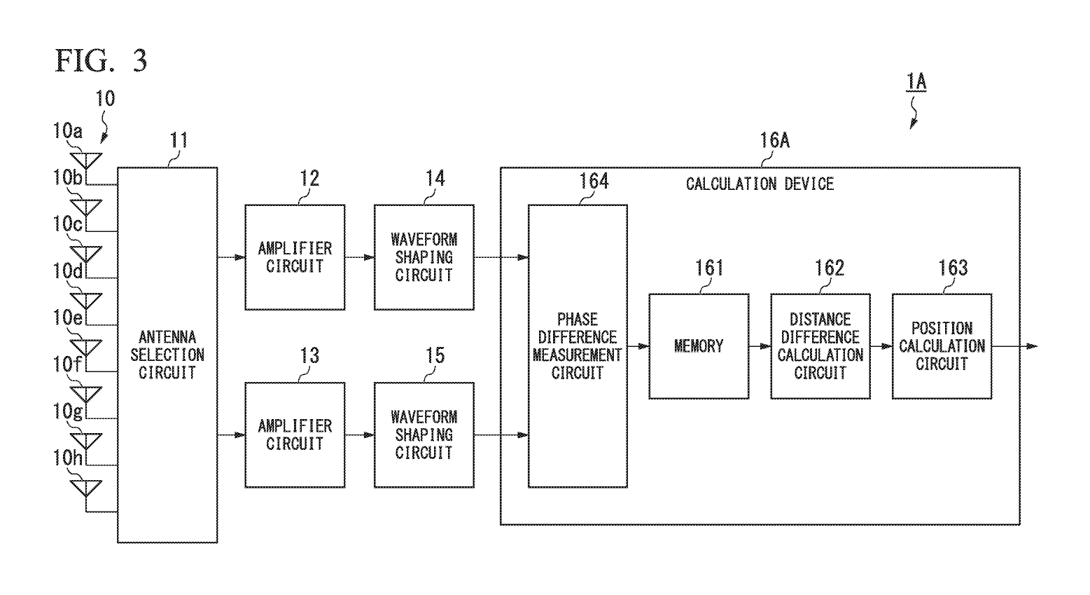

FIG. 3 shows a hardware configuration of a capsule endoscope position detection apparatus 1A which is a first modified example of the capsule endoscope position detection apparatus 1. In a configuration shown in FIG. 3, differences from the configuration shown in FIG. 1 will be described.

In the capsule endoscope position detection apparatus 1A, the calculation device 16 of the capsule endoscope position detection apparatus 1 shown in FIG. 1 is changed to a calculation device 16A. In the calculation device 16A, the time difference measurement circuit 160 of the calculation device 16 shown in FIG. 1 is changed to a phase difference measurement circuit 164. The phase difference measurement circuit 164 measures a phase difference. The phase difference is a phase difference in signals which are received by two antennas 10 when the same signals transmitted from the capsule endoscope 3 are received by two antennas 10. That is, the phase difference is a difference between the phase of the signal received by one of two antennas 10 and the phase of the signal received by the other of two antennas 10. The phase difference measurement circuit 164 measures the phase difference for each of combinations of two antennas 10 selected by the antenna selection circuit 11. The memory 161 stores the phase difference measured by the phase difference measurement circuit 164. The distance difference calculation circuit 162 calculates a distance difference on the basis of the phase difference stored in the memory 161. The configuration shown in FIG. 3 is the same as the configuration shown in FIG. 1 except for the above-described points.

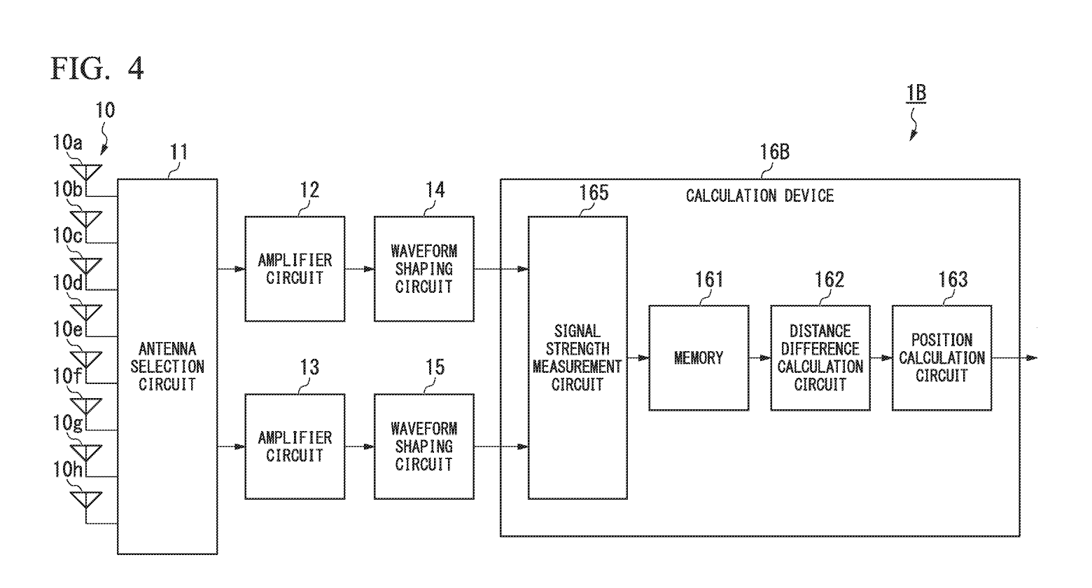

FIG. 4 shows a hardware configuration of a capsule endoscope position detection apparatus 1B which is a second modified example of the capsule endoscope position detection apparatus 1. In a configuration shown in FIG. 4, differences from the configuration shown in FIG. 1 will be described.

In the capsule endoscope position detection apparatus 1B, the calculation device 16 of the capsule endoscope position detection apparatus 1 shown in FIG. 1 is changed to a calculation device 16B. In the calculation device 16B, the time difference measurement circuit 160 of the calculation device 16 shown in FIG. 1 is changed to a signal strength measurement circuit 165. The signal strength measurement circuit 165 measures a signal strength. The signal strength is a strength of signals received by two antennas 10 when the same signals transmitted from the capsule endoscope 3 are received by two antennas 10. The signal strength which is measured by the signal strength measurement circuit 165 is based on the distance between the antenna 10 and the capsule endoscope 3. A difference in signal strength of two antennas 10 is based on the distance difference. The signal strength measurement circuit 165 measures a signal strength for each of combinations of two antennas 10 selected by the antenna selection circuit 11. The memory 161 stores the signal strength measured by the signal strength measurement circuit 165. The distance difference calculation circuit 162 calculates the distance difference on the basis of the signal strength stored in the memory 161. The configuration shown in FIG. 4 is the same as the configuration shown in FIG. 1 except for the above-described points.

The antenna selection circuit 11 may sequentially select one antenna 10 from at least four antennas 10. In this case, the antenna selection circuit 11 selects the antenna 10 so as to continuously select two antennas 10 of which the distance difference is calculated. The signal strength measurement circuit 165 sequentially measures the signal strength of the signal received by one antenna 10 selected by the antenna selection circuit 11. The distance difference calculation circuit 162 calculates the distance difference on the basis of the signal strength of each of combinations of two antennas 10 sequentially selected by the antenna selection circuit 11. In this case, the amplifier circuit 13 and the waveform shaping circuit 15 are not necessary.

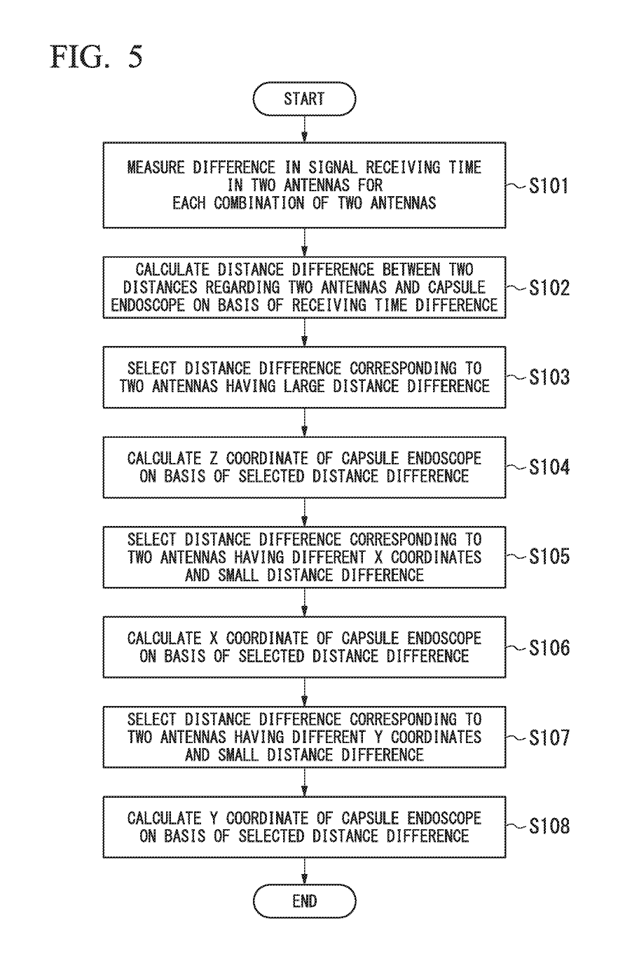

FIG. 5 shows a sequence of detecting the position of the capsule endoscope 3. Referring to FIG. 5, a method of detecting the position of the capsule endoscope 3 will be described.

(Step S101)

The antenna selection circuit 11 sequentially selects a combination of two antennas 10. The time difference measurement circuit 160 measures a difference in receiving time for the combinations of two antennas 10 selected by the antenna selection circuit 11, that is, a time difference. The measured time difference is stored in the memory 161. The number of combinations of two antennas 10 of eight antennas 10 is twenty-eight. The time difference only for a part of twenty-eight combinations may be measured.

(Step S102)

The distance difference calculation circuit 162 calculates the distance difference on the basis of the time difference stored in the memory 161.

(Step S103)

The position calculation circuit 163 selects at least three distance differences corresponding to two antennas 10 of which the distance difference is large from the distance differences calculated by the distance difference calculation circuit 162.

(Step S104)

The position calculation circuit 163 calculates the Z coordinate of the capsule endoscope 3 on the basis of at least three distance differences selected in step S103. Equation (1), Equation (2), and Equation (3) are examples of the simultaneous equations for calculating the coordinates of the capsule endoscope 3. In Equation (1) to Equation (3), (x, y, z) indicates the coordinates of the capsule endoscope 3. In Equation (1) to Equation (4), (X₁, Y₁, Z₁), (X₂, Y₂, Z₂), (X₃, Y₃, Z₃), and (X₄, Y₄, Z₄) indicate the coordinates of four antennas 10a, the antenna 10b, the antenna 10c, and the antenna 10d.

\sqrt{(x−X₁)²+(y−Y₁)²+(z−Z₁)²}−\sqrt{(x−X₂)²+(y−Y₂)²+(z−Z₂)²}=D₁₂ (1)

\sqrt{(x−X₁)²+(y−Y₁)²+(z−Z₁)²}−\sqrt{(x−X₃)²+(y−Y₃)²+(z−Z₃)²}=D₁₃ (2)

\sqrt{(x−X₁)²+(y−Y₁)²+(z−Z₁)²}−\sqrt{(x−X₄)²+(y−Y₄)²+(z−Z₄)²}−D₁₄ (3)

Equation (1) indicates a distance difference D₁₂. The distance difference D₁₂ indicates the distance difference between the first distance of the antenna 10a and the capsule endoscope 3 and the second distance of the antenna 10b and the capsule endoscope 3. Equation (2) indicates a distance difference D₁₃. The distance difference D₁₃ indicates the distance difference between the first distance of the antenna 10a and the capsule endoscope 3 and the second distance of the antenna 10c and the capsule endoscope 3. Equation (3) indicates a distance difference D₁₄. The distance difference D₁₄ indicates the distance difference between the first distance of the antenna 10a and the capsule endoscope 3 and the second distance of the antenna 10d and the capsule endoscope 3.

The simultaneous equations including Equation (1), Equation (2), and Equation (3) indicate an example of a case in which the distance difference D₁₂, the distance difference D₁₃, and the distance difference D₁₄ are larger than other distance differences. The position calculation circuit 163 calculates the coordinates (x, y, z) of the capsule endoscope 3 by solving the simultaneous equation. Among the calculated coordinates, the Z coordinate is employed as a calculation result. The number of equations constituting the simultaneous equations may be larger than three.

(Step S105)

The position calculation circuit 163 selects at least three distance differences corresponding to two antennas 10 of which the X coordinates are different and the distance difference is small from the distance differences calculated by the distance difference calculation circuit 162. In two antennas 10, the X coordinates may be different and the Y coordinates may be the same.

(Step S106)

The position calculation circuit 163 calculates the X coordinate of the capsule endoscope 3 on the basis of at least three distance differences selected in step S105. The X coordinate calculation method is the same as the Z coordinate calculation method. That is, the position calculation circuit 163 calculates the coordinates (x, y, z) of the capsule endoscope 3 by solving the simultaneous equations for at least three distance differences selected in step S105. Among the calculated coordinates, the X coordinate is employed as a calculation result.

(Step S107)

The position calculation circuit 163 selects at least three distance differences corresponding to two antennas 10 of which the Y coordinates are different and the distance difference is small from the distance differences calculated by the distance difference calculation circuit 162. In two antennas 10, the Y coordinates may be different and the X coordinates may be the same.

(Step S108)

The position calculation circuit 163 calculates the Y coordinate of the capsule endoscope 3 on the basis of at least three distance differences selected in step S107. The Y coordinate calculation method is the same as the Z coordinate calculation method. That is, the position calculation circuit 163 calculates the coordinates (x, y, z) of the capsule endoscope 3 by solving the simultaneous equations for at least three distance differences selected in step S107. Among the calculated coordinates, the Y coordinate is employed as a calculation result.

The Z coordinate calculated in step S104, the X coordinate calculated in step S106, and the Y coordinate calculated in step S108 constitute the estimated position of the capsule endoscope 3.

In the process shown in FIG. 5, the Z coordinate is calculated, the X coordinate is calculated, and then the Y coordinate is calculated. The sequence of calculating the coordinates is not limited thereto. The sequence of calculating the X coordinate, the Y coordinate, and the Z coordinate may be arbitrarily set.

As described above, the capsule endoscope position detection method includes the first step, the second step, and the third step. In the first step (step S101 and step S102), the calculation device 16 (the time difference measurement circuit 160 and the distance difference calculation circuit 162) calculates the distance difference between the first distance and the second distance on the basis of the signals wirelessly transmitted from the capsule endoscope 3 inside the subject 2 and received by two antennas 10 of at least four antennas 10. The first distance is a distance between one of two antennas 10 and the capsule endoscope 3. The second distance is a distance between the other of two antennas 10 and the capsule endoscope 3. In the first step, the distance difference for each of at least four combinations of two antennas 10 is calculated. In the second step (step S103), the calculation device 16 (the position calculation circuit 163) selects at least three distance differences from the distance differences measured in the first step. In the third step (step S104), the calculation device 16 (the position calculation circuit 163) calculates the Z coordinate of the capsule endoscope 3 on the basis of the distance differences selected in the second step.

As will be described later, the capsule endoscope position detection method according to the embodiment of the present invention can improve estimation accuracy of the Z coordinate of the capsule endoscope 3.

The capsule endoscope position detection method may further include a fourth step, a fifth step, and a sixth step. In the fourth step (step S101 and step S102), the calculation device 16 (the time difference measurement circuit 160 and the distance difference calculation circuit 162) calculates the distance difference on the basis of the signals transmitted from the capsule endoscope 3 and received by two antennas 10 of which the X coordinates are different among at least four antennas 10. In the fourth step, the distance difference for each of at least four combinations of two antennas 10 is calculated. In the fifth step (step S105), the calculation device 16 (the position calculation circuit 163) selects at least three distance differences from the distance differences selected in the fourth step. In the sixth step (step S106), the calculation device 16 (the position calculation circuit 163) calculates the X coordinate of the capsule endoscope 3 on the basis of the distance differences selected in the fifth step.

As will be described later, the capsule endoscope position detection method according to the embodiment of the present invention can improve estimation accuracy of the X coordinate of the capsule endoscope 3 by including the fourth to sixth steps.

In the fourth step for calculating the X coordinate, the calculation device 16 may calculate the distance difference on the basis of the signals received by two antennas 10 of which the X coordinates are different and the Y coordinates are the same among at least four antennas 10. Accordingly, estimation accuracy of the X coordinate of the capsule endoscope 3 is further improved.

In the fourth step for calculating the X coordinate, the antenna selection circuit 11 sequentially selects two antennas 10 of which the X coordinates are different from at least four antennas 10. The antenna selection circuit 11 may sequentially select two antennas 10 of which the X coordinates are different and the Y coordinates are the same from at least four antennas 10.

The step in which the calculation device 16 calculates the distance difference on the basis of the signals received by two antennas 10 of which the X coordinates are the same is not included in the fourth step for calculating the X coordinate. When the distance difference corresponding to the combination of two antennas 10 of which the X coordinates are the same is calculated, the distance difference is not selected in the fifth step for calculating the X coordinate.

In the fourth step (step S101 and step S102), the calculation device 16 (the time difference measurement circuit 160 and the distance difference calculation circuit 162) may calculate the distance difference on the basis of the signals transmitted from the capsule endoscope 3 and received by two antennas 10 of which the Y coordinates are different among at least four antennas 10. In the fifth step (step S107), the calculation device 16 (the position calculation circuit 163) may select at least three distance differences from the distance differences calculated in the fourth step. In the sixth step (step S108), the calculation device 16 (the position calculation circuit 163) may calculate the Y coordinate of the capsule endoscope 3 on the basis of the distance differences selected in the fifth step.

As will be described later, the capsule endoscope position detection method according to the embodiment of the present invention can improve estimation accuracy of the Y coordinate of the capsule endoscope 3 by including the fourth to sixth steps.

In the fourth step for calculating the Y coordinate, the calculation device 16 may calculate the distance difference on the basis of the signals received by two antennas 10 of which the Y coordinates are different and the X coordinates are the same from at least four antennas 10. Accordingly, estimation accuracy of the Y coordinate of the capsule endoscope 3 is further improved.

In the fourth step for calculating the Y coordinate, the antenna selection circuit 11 sequentially selects two antennas 10 of which the Y coordinates are different from at least four antennas 10. The antenna selection circuit 11 may sequentially select two antennas 10 of which the Y coordinates are different and the X coordinates are the same from at least four antennas 10.

The step in which the calculation device 16 calculates the distance difference on the basis of the signals received by two antennas 10 of which the Y coordinates are the same is not included in the fourth step for calculating the Y coordinate. When the distance difference corresponding to the combination of two antennas 10 of which the Y coordinates are the same is calculated, the distance difference is not selected in the fifth step for calculating the Y coordinate.

The first step may include the fourth step. That is, the first step may include a first sub-step and a second sub-step. In the first sub-step, the calculation device 16 calculates the distance difference on the basis of the signals transmitted from the capsule endoscope 3 and received by two antennas 10 of which the X coordinates or the Y coordinates are the same among at least four antennas 10. The second sub-step is the fourth step. The first step and the fourth step may be performed as different steps.

An important object of the embodiment of the present invention is to improve estimation accuracy of the Z coordinate of the capsule endoscope 3. It is not essential to improve the estimation accuracy of the X coordinate and the Y coordinate of the capsule endoscope 3. Accordingly, the fourth to sixth steps are not steps essential to the capsule endoscope position detection method according to the embodiment of the present invention. The X coordinate, the Y coordinate, and the Z coordinate calculated in step S104 may constitute the estimated position of the capsule endoscope 3.

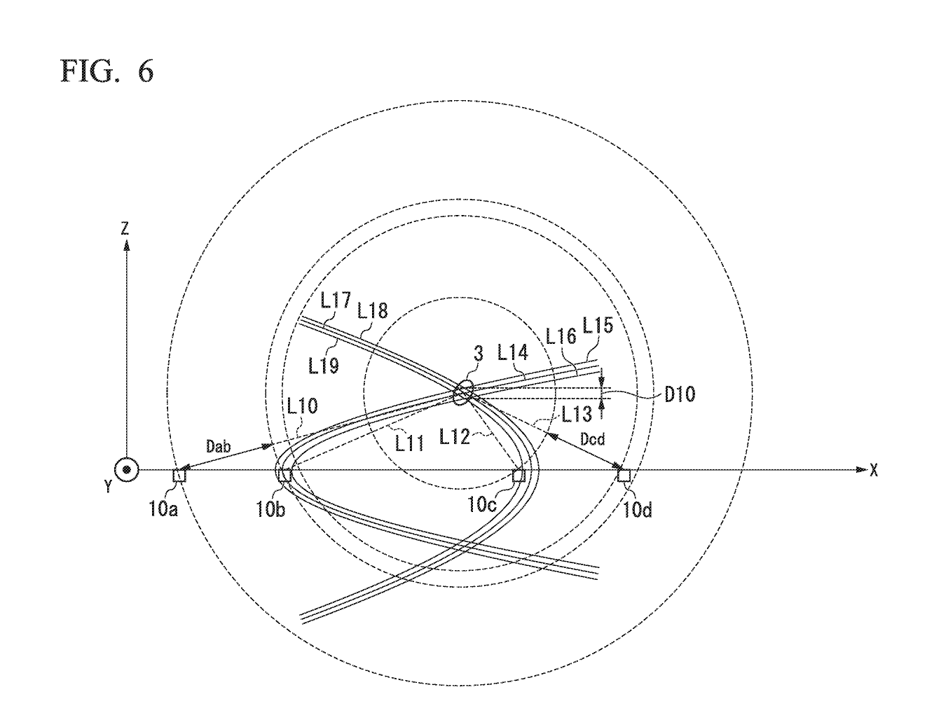

Estimation accuracy of the Z coordinate will be described. FIG. 6 shows a case in which estimation accuracy of the Z coordinate is high. FIG. 6 shows the X axis, the Y axis, and the Z axis. The X direction is the right direction in FIG. 6. The Y direction is the front direction in FIG. 6. The Z direction is the up direction in FIG. 6. In FIG. 6, the antenna 10a, the antenna 10b, the antenna 10c, and the antenna 10d disposed on the XY plane are shown.

A length of a line L10 connecting the antenna 10a and the capsule endoscope 3 shows the distance between the antenna 10a and the capsule endoscope 3. A length of a line L11 connecting the antenna 10b and the capsule endoscope 3 shows the distance between the antenna 10b and the capsule endoscope 3. A length of a line L12 connecting the antenna 10c and the capsule endoscope 3 shows the distance between the antenna 10c and the capsule endoscope 3. A length of a line L13 connecting the antenna 10d and the capsule endoscope 3 shows the distance between the antenna 10d and the capsule endoscope 3.

A distance difference Dab corresponding to the combination of the antenna 10a and the antenna 10b indicates a difference between the length of the line L10 and the length of the line L11. A hyperbola L14 is a locus of a point of which distances from the antenna 10a and the antenna 10b are uniform (Dab). The position of the capsule endoscope 3 is located on the hyperbola L14. There is a case in which the distance difference includes an error due to the influence of the receiving time measurement accuracy or the like. When the distance difference includes an error, the hyperbola L14 becomes a hyperbola L15 or a hyperbola L16.

A distance difference Dcd corresponding to the combination of the antenna 10c and the antenna 10d indicates a difference between the length of the line L12 and the length of the line L13. The hyperbola L17 is a locus of a point of which distances from the antenna 10c and the antenna 10d are uniform (Dcd). A position of the capsule endoscope 3 is located on the hyperbola L17. When the distance difference includes an error, the hyperbola L17 becomes a hyperbola L18 or a hyperbola L19.

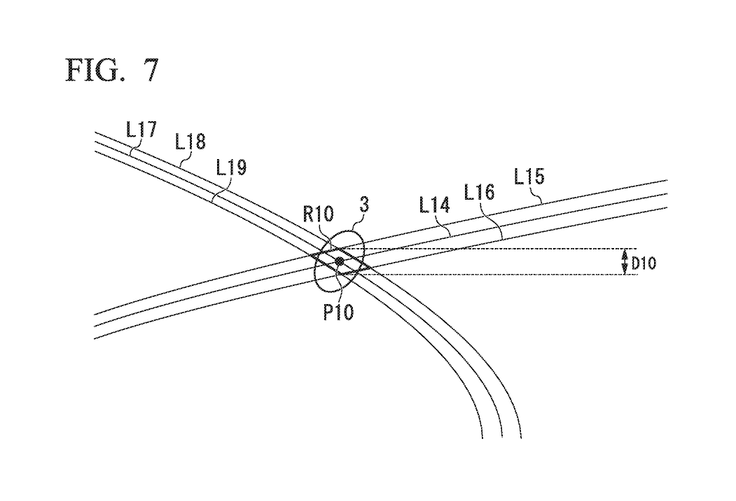

FIG. 7 is an enlarged view of a region in the periphery of the capsule endoscope 3 in FIG. 6. A position P10 of the capsule endoscope 3 is estimated as a position within a region R10. The region R10 is a region surrounded by the hyperbola L15, the hyperbola L16, the hyperbola L18, and the hyperbola L19. A distance D10 is a distance between a position of which the Z coordinate is maximal and a position of which the Z coordinate is minimal in the region R10. The distance D10 shows estimation accuracy of the Z coordinate of the capsule endoscope 3.

When the distance difference corresponding to the combination of two antennas 10 is large, the hyperbola is substantially perpendicular to the Z axis. When the hyperbola is substantially perpendicular to the Z axis, the distance D10 is small. That is, a change in a calculation result of the Z coordinate for an error of the distance difference is small.

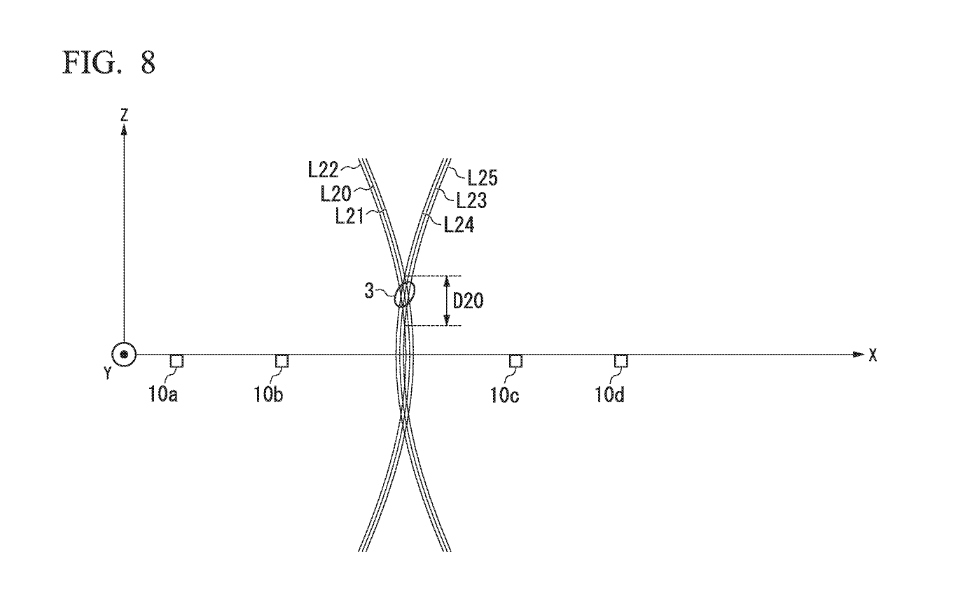

FIG. 8 shows a case in which estimation accuracy of the Z coordinate is low. FIG. 8 shows the X axis, the Y axis, and the Z axis. The X direction is a right direction in FIG. 8. The Y direction is a front direction in FIG. 8. The Z direction is an up direction in FIG. 8. In FIG. 8, the antenna 10a, the antenna 10b, the antenna 10c, and the antenna 10d disposed on the XY plane are shown.

A hyperbola L20 is a locus of a point of which distances from the antenna 10b and the antenna 10d are uniform. A position of the capsule endoscope 3 is located on the hyperbola L20. When the distance difference includes an error, the hyperbola L20 becomes a hyperbola L21 or a hyperbola L22.

A hyperbola L23 is a locus of a point of which distances from the antenna 10b and the antenna 10c are uniform. A position of the capsule endoscope 3 is located on the hyperbola L23. When the distance difference includes an error, the hyperbola L23 becomes a hyperbola L24 or a hyperbola L25.

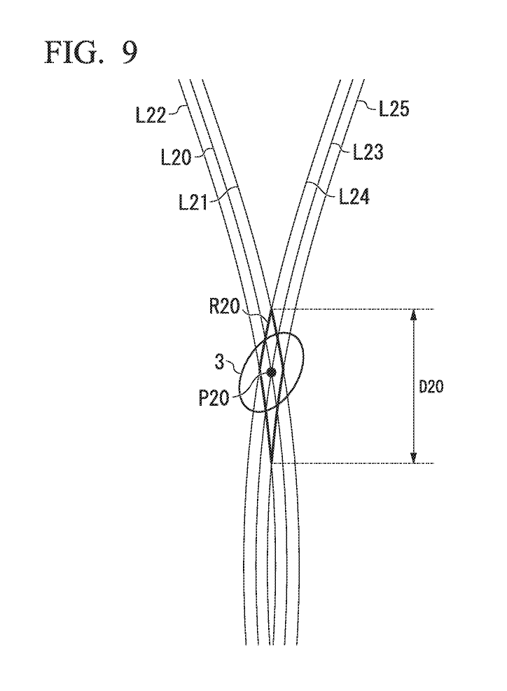

FIG. 9 is an enlarged view of a region in the periphery of the capsule endoscope 3 in FIG. 8. A position P20 of the capsule endoscope 3 is estimated as a position within a region R20. The region R20 is a region which is surrounded by the hyperbola L21, the hyperbola L22, the hyperbola L24, and the hyperbola L25. A distance D20 is a distance between a position of which the Z coordinate is maximal and a position of which the Z coordinate is minimal in the region R20. The distance D20 shows estimation accuracy of the Z coordinate of the capsule endoscope 3.

When the distance difference corresponding to the combination of two antennas 10 is small, the hyperbola is substantially parallel to the Z axis. When the hyperbola is substantially parallel to the Z axis, the distance D20 is large. That is, a change in a calculation result of the Z coordinate for an error of the distance difference is large.

The distance D10 is smaller than the distance D20. That is, the calculation device 16 can estimate the Z coordinate of the capsule endoscope 3 with high accuracy by calculating the Z coordinate of the capsule endoscope 3 using larger distance differences.

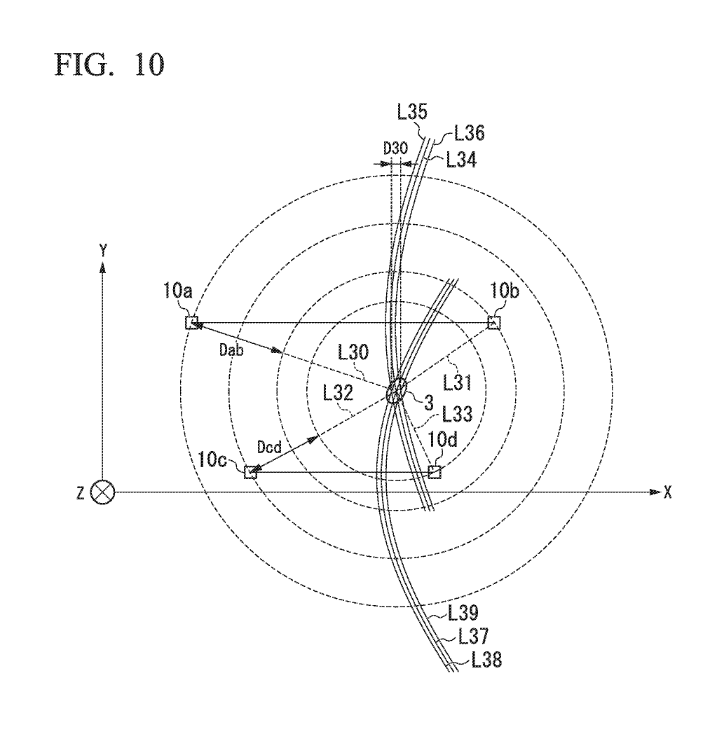

Estimation accuracy of the X coordinate will be described. FIG. 10 shows a case in which estimation accuracy of the X coordinate is high. FIG. 10 shows the X axis, the Y axis, and the Z axis. The X direction is a right direction in FIG. 10. The Y direction is an up direction in FIG. 10. The Z direction is a back direction in FIG. 10. In FIG. 10, the antenna 10a, the antenna 10b, the antenna 10c, and the antenna 10d disposed on the XY plane are shown. The X coordinates of the antenna 10a and the antenna 10b are different and the Y coordinates of the antenna 10a and the antenna 10b are the same. The X coordinates of the antenna 10c and the antenna 10d are different and the Y coordinates of the antenna 10c and the antenna 10d are the same.

A length of a line L30 connecting the antenna 10a and the capsule endoscope 3 shows the distance between the antenna 10a and the capsule endoscope 3. A length of a line L31 connecting the antenna 10b and the capsule endoscope 3 shows the distance between the antenna 10b and the capsule endoscope 3. A length of a line L32 connecting the antenna 10c and the capsule endoscope 3 shows the distance between the antenna 10c and the capsule endoscope 3. A length of a line L33 connecting the antenna 10d and the capsule endoscope 3 shows the distance between the antenna 10d and the capsule endoscope 3.

The distance difference Dab corresponding to the combination of the antenna 10a and the antenna 10b is a difference between the length of the line L30 and the length of the line L31. The hyperbola L34 is a locus of a point of which distances from the antenna 10a and the antenna 10b are uniform (Dab). A position of the capsule endoscope 3 is located on the hyperbola L34. When the distance difference includes an error, the hyperbola L34 becomes a hyperbola L35 or a hyperbola L36.

The distance difference Dcd corresponding to the combination of the antenna 10c and the antenna 10d is a difference between the length of the line L32 and the length of the line L33. A hyperbola L37 is a locus of a point of which distances from the antenna 10c and the antenna 10d are uniform (Dcd). A position of the capsule endoscope 3 is located on the hyperbola L37. When the distance difference includes an error, the hyperbola L37 becomes a hyperbola L38 or a hyperbola L39.

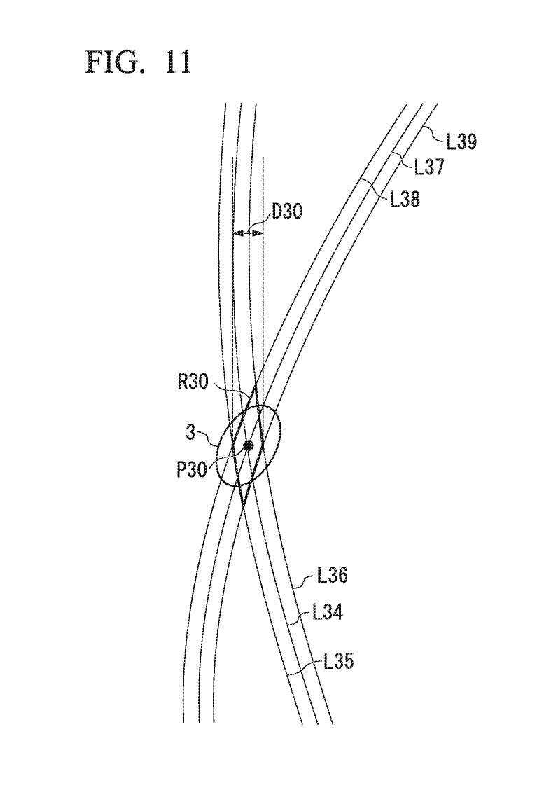

FIG. 11 is an enlarged view of a region in the periphery of the capsule endoscope 3 in FIG. 10. A position P30 of the capsule endoscope 3 is estimated as a position within a region R30. The region R30 is a region surrounded by the hyperbola L35, the hyperbola L36, the hyperbola L38, and the hyperbola L39. A distance D30 is a distance between a position of which the X coordinate is maximal and a position of which the X coordinate is minimal in the region R30. The distance D30 shows estimation accuracy of the X coordinate of the capsule endoscope 3.

When the distance difference corresponding to the combination of two antennas 10 is small, the hyperbola is substantially perpendicular to the X axis. When the hyperbola is substantially perpendicular to the X axis, the distance D30 is small. That is, a change in a calculation result of the X coordinate for an error of the distance difference is small.

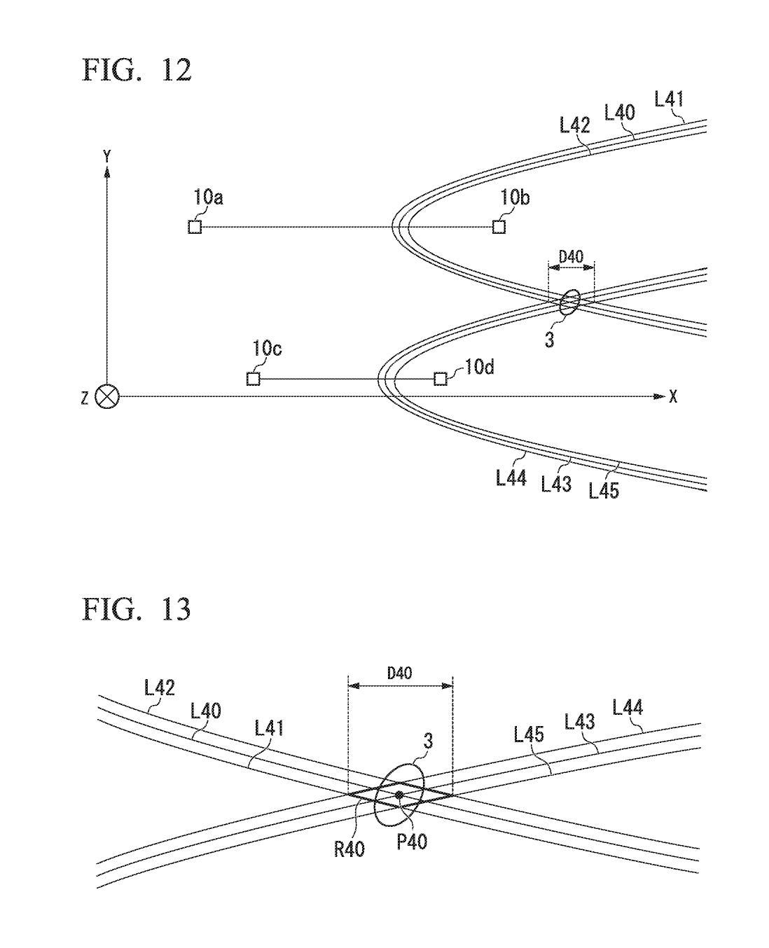

FIG. 12 shows a case in which estimation accuracy of the X coordinate is low. FIG. 12 shows the X axis, the Y axis, and the Z axis. The X direction is a right direction in FIG. 12. The Y direction is an up direction in FIG. 12. The Z direction is a back direction in FIG. 12. In FIG. 12, the antenna 10a, the antenna 10b, the antenna 10c, and the antenna 10d disposed on the XY plane are shown.

A hyperbola L40 is a locus of a point of which distances from the antenna 10a and the antenna 10b are uniform. A position of the capsule endoscope 3 is located on the hyperbola L40. When the distance difference includes an error, the hyperbola L40 becomes a hyperbola L41 or a hyperbola L42.

A hyperbola L43 is a locus of a point of which distances from the antenna 10c and the antenna 10d are uniform. A position of the capsule endoscope 3 is located on the hyperbola L43. When the distance difference includes an error, the hyperbola L43 becomes a hyperbola L44 or a hyperbola L45.

FIG. 13 is an enlarged view of a region in the periphery of the capsule endoscope 3 in FIG. 12. A position P40 of the capsule endoscope 3 is estimated as a position within a region R40. The region R40 is a region surrounded by the hyperbola L41, the hyperbola L42, the hyperbola L44, and the hyperbola L45. A distance D40 is a distance between a position of which the X coordinate is maximal and a position of which the X coordinate is minimal in the region R40. The distance D40 shows estimation accuracy of the X coordinate of the capsule endoscope 3.

When the distance difference corresponding to the combination of two antennas 10 is large, the hyperbola is substantially parallel to the X axis. When the hyperbola is substantially parallel to the X axis, the distance D40 is large. That is, a change in a calculation result of the X coordinate for an error of the distance difference is large.

The distance D30 is smaller than the distance D40. That is, the calculation device 16 can estimate the X coordinate of the capsule endoscope 3 with high accuracy by calculating the X coordinate of the capsule endoscope 3 using smaller distance differences. The X coordinates of two antennas 10 used to calculate the distance difference are different. When the Y coordinates of two antennas 10 used to calculate the distance difference are the same, estimation accuracy of the X coordinate of the capsule endoscope 3 is improved more than a case in which the Y coordinates of two antennas 10 are different.

Estimation accuracy of the Y coordinate is the same as estimation accuracy of the X coordinate. That is, the calculation device 16 can estimate the Y coordinate of the capsule endoscope 3 with high accuracy by calculating the Y coordinate of the capsule endoscope 3 using smaller distance differences. The Y coordinates of two antennas 10 used to calculate the distance difference are different. When the X coordinates of two antennas 10 used to calculate the distance difference are the same, estimation accuracy of the Y coordinate is improved more than a case in which the X coordinates of two antennas 10 are different.

In order to improve estimation accuracy of the X coordinate, at least four antennas 10 are disposed so that the X coordinates of the antennas 10 are different. In order to improve estimation accuracy of the Y coordinate, at least four antennas 10 are disposed so that the Y coordinates of the antennas 10 are different.

The distance difference selection method in step S103 will be described. For example, in the second step (step S103), the calculation device 16 (the position calculation circuit 163) selects the three distance differences from the top in the order of magnitude of distance difference for at least four combinations.

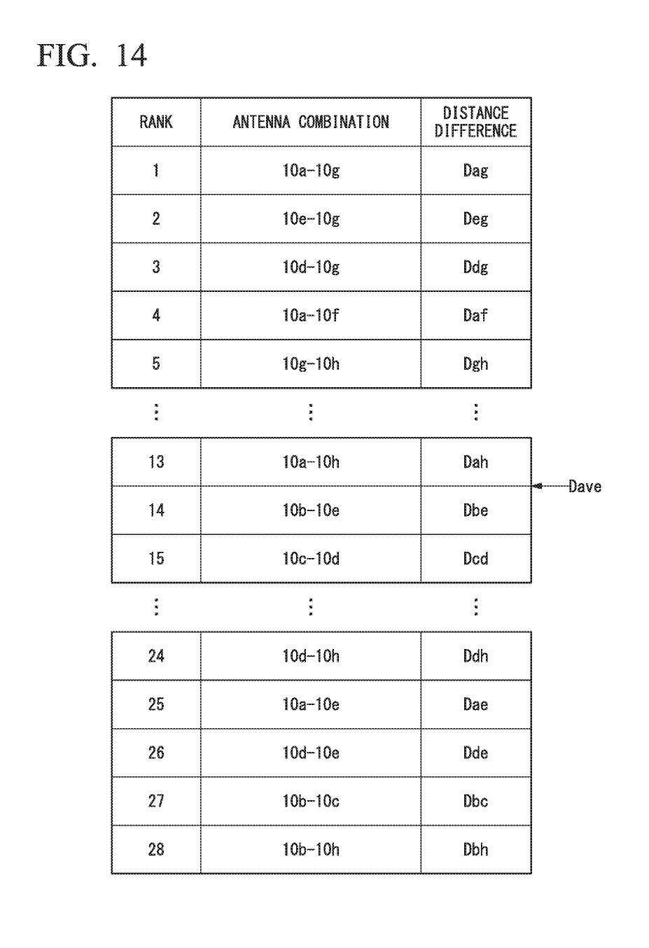

FIG. 14 shows the distance difference for each combination of two antennas 10. The distance differences for each of twenty-eight combinations are arranged in the order of magnitude. In FIG. 14, the rank, the combination of two antennas 10, and the distance difference are correlated with one another. The lower-ranking distance difference is larger. The combination of two antennas 10 is indicated by the reference numerals from each of the antenna 10a to the antenna 10h. For example, the combination of two antennas with the highest ranking includes the antenna 10a and the antenna 10g. The distance difference is identified by the character corresponding to each of the antenna 10a to the antenna 10h. For example, the distance difference corresponding to the antenna 10a and the antenna 10g is Dag. In the second step (step S103), the calculation device 16 selects the maximum distance difference Dag, the second largest distance difference Deg, and the third largest distance difference Ddg. That is, in the second step (step S103), the calculation device 16 selects the first-rank distance difference Dag, the second-rank distance difference Deg, and the third-rank distance difference Ddg.

In the second step (step S103), the calculation device 16 (the position calculation circuit 163) may select at least three distance differences in which the distance difference is equal to or larger than a predetermined value among the distance differences for at least four combinations. For example, the predetermined value is an average value or a median value of the distance differences for at least four combinations.

The average value of twenty-eight distance differences shown in FIG. 14 is Dave. The average value Dave is larger than the fourteenth-rank distance difference Dbe and is smaller than the thirteenth-rank distance difference Dah. For example, the calculation device 16 selects at least three distance differences from the first-rank to thirteenth-rank distance differences.

The median value of twenty-eight distance differences shown in FIG. 14 is an average value of the fourteenth-rank distance difference Dbe and the fifteenth-rank distance difference Dcd. For example, the calculation device 16 selects at least three distance differences from the first-rank to fourteenth-rank distance differences.

The predetermined value is not limited to the average value and the median value. The predetermined value may be a value other than the average value and the median value.

A first average value may be larger than a second average value. The first average value is an average value of at least three distance differences selected in the second step (step S103). The second average value is an average value of the distance differences for at least four combinations.

For example, the second average value is the average value Dave of twenty-eight distance differences shown in FIG. 14. The calculation device 16 selects at least three distance differences so that the first average value becomes larger than the second average value. The distance difference shown in step S103 may include the distance difference smaller than the first average value.

The distance difference selection method in step S105 and step S107 will be described. For example, in the fifth step (step S105 or step S107), the calculation device 16 (the position calculation circuit 163) selects the three small distance differences from the bottom in the order of magnitude of distance difference for at least four combinations. The three small distance differences include the minimum distance difference, the second smallest distance difference, and the third smallest distance difference.

In the fifth step, the calculation device 16 (the position calculation circuit 163) may select at least three distance differences in which the distance difference is equal to or smaller than a predetermined value among the distance differences for at least four combinations. For example, the predetermined value is an average value or a median value of the distance differences for at least four combinations.

A third average value may be smaller than a fourth average value. The fourth average value is an average value of the distance differences for at least four combinations. The third average value is an average value of at least three distance differences selected in the fifth step.

The calculation device 16 selects at least three distance differences so that the third average value becomes smaller than the fourth average value. The distance difference selected in step S105 or step S107 may include the distance difference larger than the third average value.

As described above, in the first step (step S101 and step S102) or the fourth step (step S101 and step S102), the calculation device 16 (the time difference measurement circuit 160 or the phase difference measurement circuit 164) calculates the distance difference on the basis of the time difference or the phase difference. The time difference is a difference in time when the same signals transmitted from the capsule endoscope 3 are received by two antennas 10. The phase difference is a phase difference in signals received by two antennas 10 when the same signals transmitted from the capsule endoscope 3 are received by two antennas 10.

In the first step (step S101 and step S102) or the fourth step (step S101 and step S102), the calculation device 16 (the time difference measurement circuit 160 or the phase difference measurement circuit 164) may calculate the distance difference on the basis of the signal strength of the signal received by two antennas 10 when the same signals transmitted from the capsule endoscope 3 are received by two antennas 10.

As described above, the calculation device 16 can estimate the Z coordinate of the capsule endoscope 3 with high accuracy by calculating the Z coordinate of the capsule endoscope 3 using larger distance differences. That is, estimation accuracy of the Z coordinate of the capsule endoscope 3 is improved.

The calculation device 16 can estimate the X coordinate of the capsule endoscope 3 with high accuracy by calculating the X coordinate of the capsule endoscope 3 using smaller distance differences. That is, estimation accuracy of the X coordinate of the capsule endoscope 3 is improved.

The calculation device 16 can estimate the Y coordinate of the capsule endoscope 3 with high accuracy by calculating the Y coordinate of the capsule endoscope 3 using smaller distance differences. That is, estimation accuracy of the Y coordinate of the capsule endoscope 3 is improved.

While preferred embodiments of the invention have been described and shown above, it should be understood that these are exemplars of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A capsule endoscope position detection method using at least four antennas configured to receive signals wirelessly transmitted from a capsule endoscope inside a subject, the method comprising:
a first step in which a calculation device calculates a distance difference between a first distance and a second distance on the basis of the signals wirelessly transmitted from the capsule endoscope and received by two of the at least four antennas, the at least four antennas being disposed on a first plane, the first plane being substantially parallel to a second plane, the second plane including X and Y axes in an orthogonal coordinate system including X, Y, and Z axes, the first distance being a distance between one of the two antennas and the capsule endoscope, the second distance being a distance between the other of the two antennas and the capsule endoscope, and the distance difference being calculated for each of at least four combinations of the two antennas;
a second step in which the calculation device selects at least three distance differences on the basis of the magnitude of the distance differences calculated in the first step; and
a third step in which the calculation device calculates a Z coordinate of the capsule endoscope on the basis of the distance differences selected in the second step.

2. The capsule endoscope position detection method according to claim 1,
wherein in the second step, the calculation device selects three large distance differences from the top in the order of magnitude of the distance differences for the at least four combinations.

3. The capsule endoscope position detection method according to claim 1,
wherein in the second step, the calculation device selects at least three distance differences which are equal to or larger than a predetermined value among the distance differences for the at least four combinations.

4. The capsule endoscope position detection method according to claim 3,
wherein the predetermined value is an average value of the distance differences for the at least four combinations or a median value of the distance differences for the at least four combinations.

5. The capsule endoscope position detection method according to claim 1,
wherein a first average value is larger than a second average value, the first average value is an average value of the at least three distance differences selected in the second step, and the second average value is an average value of the distance differences for the at least four combinations.

6. The capsule endoscope position detection method according to claim 1, further comprising:
a fourth step in which the calculation device calculates the distance difference on the basis of the signals transmitted from the capsule endoscope and received by the two antennas of which X coordinates are different among the at least four antennas, the distance difference being calculated for each of at least four combinations of the two antennas;
a fifth step in which the calculation device selects at least three distance differences among the distance differences calculated in the fourth step; and
a sixth step in which the calculation device calculates an X coordinate of the capsule endoscope on the basis of the distance differences selected in the fifth step.

7. The capsule endoscope position detection method according to claim 1, further comprising:
a fourth step in which the calculation device calculates the distance difference on the basis of the signals transmitted from the capsule endoscope and received by the two antennas of which Y coordinates are different among at least four antennas, the distance difference being calculated for each of at least four combinations of the two antennas;
a fifth step in which the calculation device selects at least three distance differences from the distance differences calculated in the fourth step; and
a sixth step in which the calculation device calculates a Y coordinate of the capsule endoscope on the basis of the distance differences selected in the fifth step.

8. The capsule endoscope position detection method according to claim 6,
wherein in the fifth step, the calculation device selects three small distance differences from the bottom in the order of magnitude of the distance differences for the at least four combinations.

9. The capsule endoscope position detection method according to claim 7,
wherein in the fifth step, the calculation device selects three small distance differences from the bottom in the order of magnitude of the distance differences for the at least four combinations.

10. The capsule endoscope position detection method according to claim 6,
wherein in the fifth step, the calculation device selects at least three distance differences in which the distance difference is equal to or smaller than a predetermined value among the distance differences for the at least four combinations.

11. The capsule endoscope position detection method according to claim 7,
wherein in the fifth step, the calculation device selects at least three distance differences in which the distance difference is equal to or smaller than a predetermined value among the distance differences for the at least four combinations.

12. The capsule endoscope position detection method according to claim 10,
wherein the predetermined value is an average value of the distance differences for the at least four combinations or a median value of the distance differences for the at least four combinations.

13. The capsule endoscope position detection method according to claim 11,
wherein the predetermined value is an average value of the distance differences for the at least four combinations or a median value of the distance differences for the at least four combinations.

14. The capsule endoscope position detection method according to claim 6,
wherein a third average value is smaller than a fourth average value, the third average value is an average value of the at least three distance differences selected in the fifth step, and the fourth average value is an average value of the distance differences for the at least four combinations.

15. The capsule endoscope position detection method according to claim 7,
wherein a third average value is smaller than a fourth average value, the third average value is an average value of the at least three distance differences selected in the fifth step, and the fourth average value is an average value of the distance differences for the at least four combinations.

16. The capsule endoscope position detection method according to claim 1,
wherein in the first step, the calculation device calculates the distance difference on the basis of a time difference or a phase difference, the time difference is a difference in time when the same signals transmitted from the capsule endoscope are received by the two antennas, and the phase difference is a phase difference in the signals received by the two antennas when the same signals transmitted from the capsule endoscope are received by the two antennas.

17. The capsule endoscope position detection method according to claim 6,
wherein in the fourth step, the calculation device calculates the distance difference on the basis of a time difference or a phase difference, the time difference is a difference in time when the same signals transmitted from the capsule endoscopes are received by the two antennas, and the phase difference is a phase difference in the signals received by the two antennas when the same signals transmitted from the capsule endoscope are received by the two antennas.

18. The capsule endoscope position detection method according to claim 1,
wherein in the first step, the calculation device calculates the distance difference on the basis of a signal strength of the signals received by the two antennas when the same signals transmitted from the capsule endoscope are received by the two antennas.

19. The capsule endoscope position detection method according to claim 6,
wherein in the fourth step, the calculation device calculates the distance difference on the basis of a signal strength of the signals received by the two antennas when the same signals transmitted from the capsule endoscope are received by the two antennas.

20. A capsule endoscope position detection apparatus, comprising:
at least four antennas configured to receive signals wirelessly transmitted from a capsule endoscope inside a subject;
an antenna selection circuit configured to sequentially selects one or two antennas from the at least four antennas; and
a calculation device,
wherein the at least four antennas are disposed on a first plane, the first plane is substantially parallel to a second plane, and the second plane includes X and Y axes in an orthogonal coordinate system including X, Y, and Z axes,
wherein in a first step, the calculation device calculates a distance difference between a first distance and a second distance on the basis of signals received by two antennas simultaneously or sequentially selected by the antenna selection circuit, the first distance is a distance between one of the two antennas and the capsule endoscope, the second distance is a distance between the other of the two antennas and the capsule endoscope, and the distance difference is calculated for each of at least four combinations of the two antennas,
wherein in a second step, the calculation device selects at least three distance differences in the order of magnitude of the distance differences calculated in the first step, and
wherein in a third step, the calculation device calculates a Z coordinate of the capsule endoscope on the basis of the distance differences selected in the second step.
